(19) United States
(12) Patent Application Publication
(10) Pub. No.: US 2024/0260907 A1
(43) Pub. Date: Aug. 8, 2024

(54) PREDICTING WORSENING HEART FAILURE USING INTERMITTENT NONINVASIVE BIOMARKER MEASUREMENTS

(71) Applicant: Bodyport Inc., San Francisco, CA (US)

(72) Inventors: Corey James Centen, San Francisco, CA (US); Mehmet Kivanc Ozonat, San Jose, CA (US); Sarin Narendra Patel, Union City, CA (US); Varol Burak Aydemir, Acworth, GA (US); Shayan Guhaniyogi, Portland, OR (US); Sarah Ann Smith, San Francisco, CA (US)

(21) Appl. No.: 18/431,857

(22) Filed: Feb. 2, 2024

Related U.S. Application Data
(60) Provisional application No. 63/443,008, filed on Feb. 2, 2023.

(51) Int. Cl. A61B 5/00; A61B 5/11; A61B 5/318

(52) U.S. Cl. CPC A61B 5/7275; A61B 5/1102; A61B 5/318; A61B 5/4842; A61B 5/746

(57) ABSTRACT

A cardiovascular monitoring platform receives one or more signals collected for a user of the measurement device during a time period. The cardiovascular monitoring platform extracts measurements for a plurality of biomarkers based on the one or more signals collected for the user, wherein each biomarker characterizes an aspect of cardiovascular health of the user. The cardiovascular monitoring platform applies a heart function model to the one or more signals collected by the measurement device and the plurality of biomarkers. The heart function model outputs a heart function index that characterizes a likelihood whether the user will experience a heart failure event during a future time period. The cardiovascular monitoring platform generates an alert based on the heart function index. The alert comprises a risk state of the user determined based on a comparison of the heart function index to a threshold.

### CROSS REFERENCE TO RELATED APPLICATIONS

This application claims the benefit of U.S. Provisional Application No. 63/443,008, filed on Feb. 2, 2023, which is incorporated by reference in its entirety.

### BACKGROUND

This disclosure relates generally to methods and devices for predicting a cardiovascular risk state for a user and, more specifically, to a model configured to compute a likelihood that a user will experience a future heart failure event given a combination of signals and biomarkers collected for the user.

Heart failure (HF) is a major growing health problem impacting over 65 million people worldwide. Heart failure is characterized by recurrent episodes of decompensation that frequently result in hospitalization or death. Despite advances in medical and device therapies, the number of hospitalizations for heart failure and associated readmissions remains high and represents an increasingly unsustainable financial burden. In the US alone, costs related to heart failure are expected to reach $70 billion by 2030. Remote monitoring of patients outside the hospital has the potential to detect early signs of worsening status and provide sufficient warning to deliver interventions that prevent further deterioration. The standard approaches to heart failure monitoring include daily self-weighing and implantable sensors, but each has limitations preventing its widespread adoption. Accordingly, there is a need for improved noninvasive remote monitoring solutions that prevent hospitalizations through the early prediction and management of HF events in the outpatient setting.

Conventional weight-based remote monitoring systems detect only late stages of worsening heart failure, at which point there is often insufficient time to prevent heart failure events (i.e., hospitalizations). Monitoring systems involving implantable sensors are both expensive and often offered only to users who need an invasive procedure, which severely limits the number of eligible users. Other conventional systems implement wearable sensors, such as adhesive patches, that are only effective for continuously monitoring heart failure over short periods of approximately two weeks or less.

### SUMMARY

In accordance with one or more aspects of the disclosure, a cardiovascular monitoring platform uses a measurement device including multiple sensors that record intermittent measurements (e.g., daily measurements over a 20 to 30 second window) of one or more noninvasive physiological signals that are used to calculate one or more biomarkers for a user. The one or more physiological signals include a weight measurement for the user and electrical signals collected through the feet of the user. The cardiovascular monitoring platform extracts measurements for a plurality of biomarkers based on the one or more signals collected for the user. Each biomarker characterizes an aspect of the cardiovascular health of the user.

The cardiovascular monitoring platform applies a heart function model to the one or more signals collected by the measurement device and the plurality of biomarkers. The output of the heart function model describes a likelihood that the user will experience a heart failure event within a future time period. The output generated by the heart function model may be a numerical value that is categorized into a risk category (e.g., low, elevated, or alert), which indicates the likelihood of a heart failure event, or worsening heart failure, during a future time period (e.g., the next 30 or 90 days). Additionally, the cardiovascular monitoring platform generates and transmits an alert to be used by clinicians for monitoring worsening heart failure and predicting heart failure events. The alert includes a risk state for the user determined based on a comparison of the heart function index to a threshold or preceding heart function index.

### BRIEF DESCRIPTION OF DRAWINGS

The figures use like reference numerals to identify like elements. A letter after a reference numeral, such as “104A,” indicates that the text refers specifically to the element having that particular reference numeral. A reference numeral in the text without a following letter, such as “104,” refers to any or all of the elements in the figures bearing that reference numeral.

FIG. 1 illustrates an example system environment for a cardiovascular monitoring platform 110, in accordance with one or more embodiments.

FIGS. 2A-B illustrate an example embodiment of the measurement device where the user stands on the device, in accordance with one or more embodiments.

FIG. 3 illustrates an example system architecture for a cardiovascular monitoring platform, in accordance with one or more embodiments.

FIG. 4 is a flowchart illustrating a process for determining a heart function index, according to one or more embodiments.

FIG. 5 is a flowchart illustrating a process for deriving biomarker measurements for determining a heart function index, according to one or more embodiments.

FIGS. 6A-6E illustrate example interfaces of notifications displayed to a clinician or a user based on measurements collected by the measurement device, in accordance with one or more embodiments.

The figures depict various embodiments for purposes of illustration only. One skilled in the art will readily recognize from the following description that alternative embodiments of the structures and methods illustrated herein may be employed without departing from the principles of the disclosure described herein.

### DETAILED DESCRIPTION

### Example System Environment for Cardiovascular Monitoring Platform

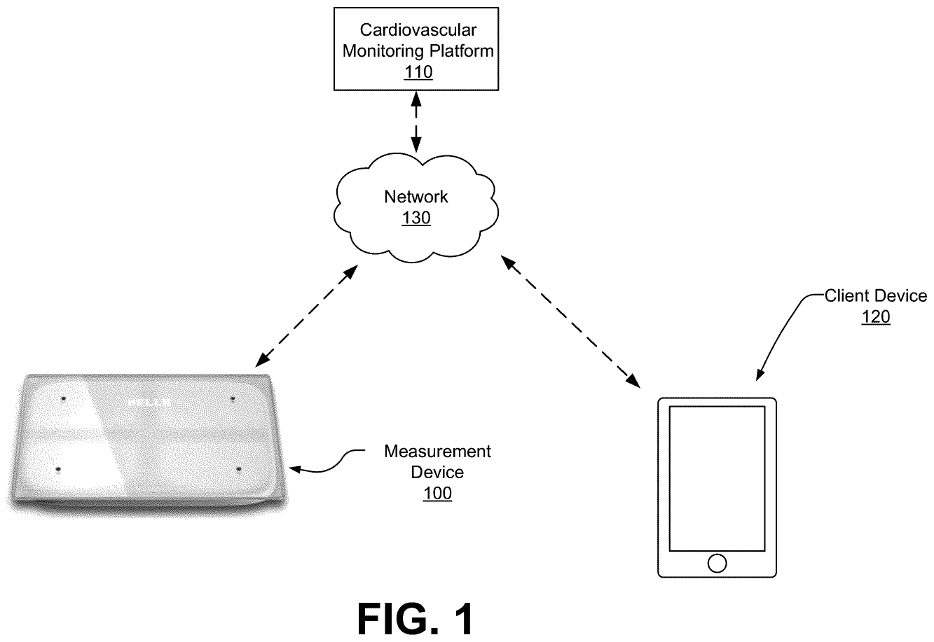

FIG. 1 illustrates an example system environment for a cardiovascular monitoring platform 110, in accordance with one or more embodiments. The system environment illustrated in FIG. 1 includes a measurement device 100, a cardiovascular monitoring platform 110, a client device 120, and a network 130. Alternative embodiments may include more, fewer, or different components from those illustrated in FIG. 1, and the functionality of each component may be divided between the components differently from the description below. For example, functionality described below as being performed by the measurement device 100 may be performed, in one or more embodiments, by the cardiovascular monitoring platform 110 or the client device 120. Similarly, functionality described below as being performed by the cardiovascular monitoring platform 110 may, in one or more embodiments, be performed by the measurement device 100 or the client device 120. Additionally, each component may perform their respective functionalities in response to a request from a human, or automatically without human intervention.

The measurement device 100 is a non-invasive, cardiovascular monitor that collects multiple signals (e.g., plethysmograph, ballistocardiograph, and electrocardiograph signals) and derives physiological biomarkers from those signals (e.g., pulse rate, body weight, and peripheral impedance). The measurement device 100 may also measure or derive cardiovascular biomarkers from the collected signals, (e.g., blood pressure, heart rate, stroke volume, ejection fraction, respiration rate, ejection force, contractility, and/or pre-ejection period). The measurement device 100 may simultaneously measure cardiovascular function, fluid status, and body weight in a single use of the device 100 by the user over a short time period (e.g., 10 to 30 seconds), for example weighing themselves daily using the measurement device 100. Such hemodynamic biomarkers measured or derived by the measurement device 100 may add context to changes in a user's weight that allow the cardiovascular monitoring platform 110 to capture earlier signs of cardiac decompensation, or worsening heart failure. In some embodiments, the cardiovascular monitoring platform 110 may use hemodynamic biomarkers instead of weight if a weight change does not occur despite worsening heart failure.

Signals and user data collected by the measurement device 100 are transmitted to the cardiovascular monitoring platform 110, which processes the collected signals into biomarkers and generates a prediction of a cardiovascular risk state for the user (also referred to as a risk state) based on the biomarkers and collected signals. As described herein, biomarkers are hemodynamic and cardiac measurements derived from characteristic features of signals measured by the measurement device 100. The cardiovascular monitoring platform 110 combines the signals measured by the measurement device 100 and biomarker measurements derived from the measured signals into a composite score characterizing the user's likelihood of a heart failure event. As described herein, a heart failure event may include, but is not limited to, a hospitalization, readmission, or emergency department visit due to heart failure, escalation of heart failure therapy or escalation of diuretics due to worsening heart failure, or objective signs or symptoms associated with worsening heart failure. As described herein, the composite index generated for a user is referred to as a “heart function index.” The composite index may also be referred to as a “composite score” or a “heart function score.” In one embodiment, the heart function index for a user is initialized at a value of zero and increases with worsening heart failure. Accordingly, the heart function index is a prediction of the likelihood that the user experiences a future heart failure event.

The cardiovascular monitoring platform 110 implements a model, referred to herein as a heart function model, to compute a heart function index for a user based on a combination of signals measured by the measurement device 100 and biomarkers derived from the measured signals. In one embodiment, the cardiovascular monitoring platform 110 inputs measurements recorded for body weight, peripheral impedance, pulse rate, or a combination thereof to the heart function model to output a heart function index for the user. Additionally, as will be further described below, the measurement device 100 may record impedance plethysmography (IPG), ballistocardiography (BCG), and electrocardiography (ECG) signals when a user operates the measurement device 100. The cardiovascular monitoring platform 110 may further process those signals into biomarker measurements to be input to the heart function model. Generally, the heart function model computes increasing heart function indices as a biomarker changes from its baseline value in the direction of worsening heart failure.

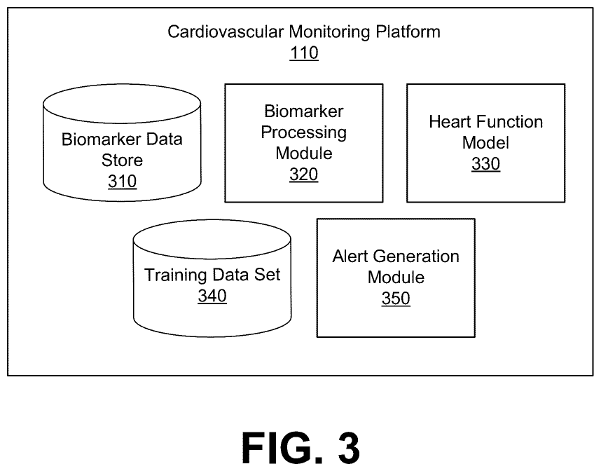

The cardiovascular monitoring platform 110 may generate alerts to both users and health care providers (e.g., physician, care team, nurse) when the heart function index increases or reaches an elevated or more severe category of heart failure. In one embodiment, the cardiovascular monitoring platform 110 may generate and transmit a report to a health care provider with the heart function index, the value of each component biomarker, a trend regarding the component biomarker, and its relative contribution to the heart function index. The generated report may be displayed via graphical user interface on a client device 120. The cardiovascular monitoring platform 110 is further described below with reference to FIGS. 3-6D.

A user can also interact with the measurement device 100 or the cardiovascular monitoring platform 110 through a client device 120. The client device 120 can be a personal or mobile computing device, such as a smartphone, a tablet, a laptop computer, or a desktop computer. In one or more embodiments, the client device 120 executes a client application that uses an application programming interface (API) to communicate with the cardiovascular monitoring platform 110 through the network 130. In one embodiment, the user of the client device is a health care provider in conjunction with standard clinical practice. In such embodiments, the health care provider may monitor changes in a user's cardiovascular state and provide feedback/update treatments based on those changes. In another embodiment, the user of the client device is a user monitoring changes in their own cardiovascular state.

The measurement device 100 and the client device 120 can communicate with the cardiovascular monitoring platform 110 via a network 130. The network 130 is a collection of computing devices that communicate via wired or wireless connections. The network 130 may include one or more local area networks (LANs) or one or more wide area networks (WANs). The network 130, as referred to herein, is an inclusive term that may refer to any or all of standard layers used to describe a physical or virtual network, such as the physical layer, the data link layer, the network layer, the transport layer, the session layer, the presentation layer, and the application layer. The network 130 may include physical media for communicating data from one computing device to another computing device, such as MPLS lines, fiber optic cables, cellular connections (e.g., 3G, 4G, 5G spectra, LTE-M), or satellites. The network 130 also may use networking protocols, such as TCP/IP, HTTP, SSH, SMS, or FTP, to transmit data between computing devices. In one or more embodiments, the network 130 may include Bluetooth or near-field communication (NFC) technologies or protocols for local communications between computing devices. The network 130 may transmit encrypted or unencrypted data.

### Example Biomarker Measurement Device

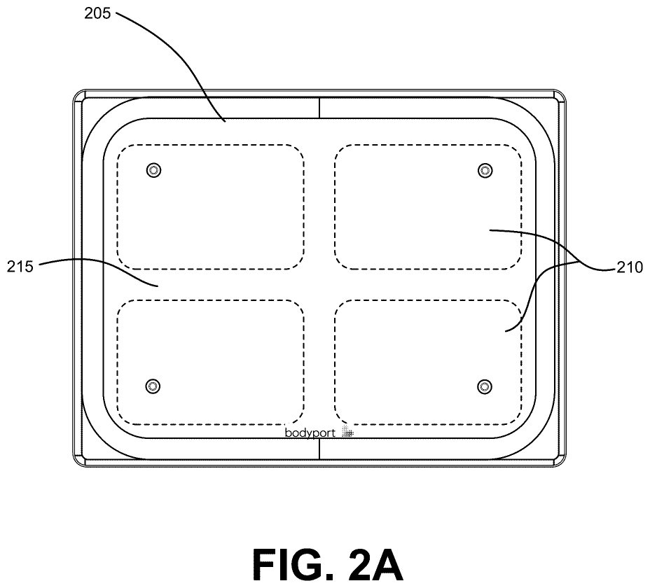

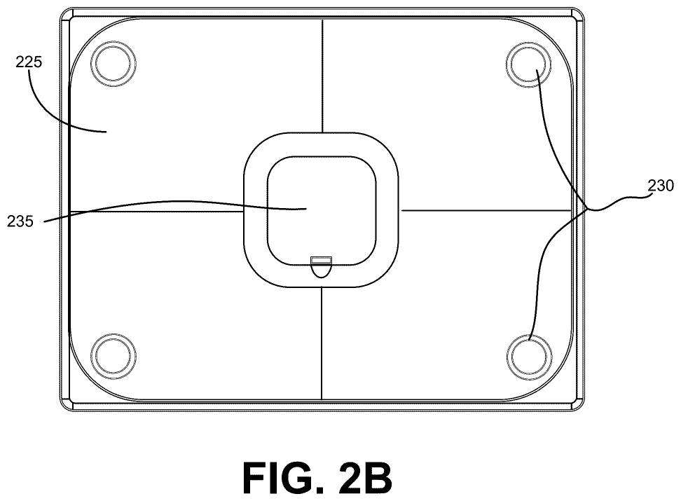

The measurement device 100 comprises a device body for supporting a user and sensors for measuring physiological signals, for example BCG, ECG, and IPG signals. In one embodiment, the measurement device 100 is a body weight scale that measures signals through the feet of a user standing on its surface. In such embodiments, the measurement device 100 comprises a physical platform on which a user stands, and one or more sensors located on the top surface of the platform to measure electrical signals, such as the baseline impedance and impedance plethysmograph (IPG) of the lower body of the user. The measurement device 100 is further described below with reference to FIGS. 2A-C. In addition to a body weight scale, the measurement device 100 may be any suitable device capable of non-invasively measuring physiological signals for a user.

FIGS. 2A-B illustrate an example embodiment of the measurement device 100 where the user stands on the measurement device 100, in accordance with one or more embodiments. The illustrated embodiment of the measurement device 100 supports the weight of a user standing on top (e.g., a body weight scale). FIG. 2A illustrates a top view of the measurement device 100, in accordance with one or more embodiments. In the illustrated embodiment, the device 100 may include a body 205 that generally houses electronic components of the device 100.

As described above, the measurement device 100 captures several signals used to derive physiological biomarkers. Accordingly, the device 100 may be configured with one or more sensors, which individually or in combination measure a biological signal. Each sensor is capable of measuring at least one signal. For example, the sensors may include piezoresistive force sensors, load cells, piezoelectric sensors, temperature sensors, electrocardiograph (ECG) sensors, bioimpedance electrodes, impedance plethysmography (IPG) sensors, optical photoplethysmography (PPG) sensors, magnetic field sensors or any other sensor capable of measuring a biological signal such as a cardiac or vascular signal.

In the illustrated embodiment, the measurement device 100 is configured with four electrical signal sensors 210, which are positioned on either side of the centerline of the device 100, for example where the user would place the heel of each foot when using the device 100. The electrical signal sensors 210 may be configured to make direct contact with the user's skin or indirect contact with the user (e.g., contact through a layer of clothing such as a sock). Each sensor 210 comprises one or more electrodes (not shown) where the user contacts the sensor. In some embodiments, the sensors 210 operate in pairs to detect a signal between the defined pairs of sensors. In the illustrated embodiments, the sensors 210 are located in four quadrants of the conductive layer 215 such that electrodes in each quadrant make contact with a foot of a user during user. A person of ordinary skill in the art would appreciate that the sensors 210 may be located elsewhere within the conductive layer 215. For example, the sensors 210 may be located to detect contact with different portions of the user's feet. In some embodiments, multiple sensors 210 may be placed at multiple different positions on the device 100, for example to accommodate ranges in feet sizes and imprecise placement of the user's feet.

FIG. 2B is a bottom view of the measurement device 100, in accordance with one or more embodiments. In the illustrated embodiment, the measurement device 100 is configured with multiple force sensors 230 on the bottom surface 225. As described herein, force sensors 230 may also be referred to as “load cells.” The force sensors 230 are used to measure the user's body weight and center of pressure, which provides data describing the motion (e.g., sway) of the user while standing on the scale. Accordingly, each force sensor 230 is positioned to detect the force exerted by the user on the device body when the device 100 is in use. In the illustrated embodiment, each force sensor 230 is positioned at a corner of the device 100. The force sensors 230 are arranged in a manner to ensure an accurate and precise measurement of the force exerted on the device 100 due to cardiovascular events, such as ejection of blood into the aorta, within the user's body. A person having ordinary skill in the art would appreciate that the force sensors 230 may be arranged in any suitable manner to detect the force exerted by the user on the device body. For example, in some embodiments, the force sensors 230 may be positioned within the device body.

The device 100 may be powered by a power source such as a battery or an external power source. In the illustrated embodiment, the device 100 may include a battery receptacle 235 for receiving a battery (e.g., a rechargeable battery or a standard disposable battery). In some examples, the device 100 may additionally or alternatively include a connector or port for receiving power from an external power source (e.g., a wall socket).

In addition, the measurement device 100 may be further configured to simultaneously record signals characterizing the mechanical function of the heart (e.g., ballistocardiograph (BCG) signals), electrical function of the heart (e.g., electrocardiograph (ECG) signals), and/or vascular function of the circulatory system (e.g., impedance plethysmograph (IPG) signals) using the combination of load cells and electrodes described above. In some embodiments, the cardiovascular monitoring platform 110 temporally aligns signals measured by the measurement device 100 so that the cardiovascular monitoring platform 110 may derive biomarker measurements based on temporal relationships between the measured signals.

IPG measurements characterize the fluid levels and pulsatile blood flow of a user based on small changes in the electrical impedance of the lower extremities. Described differently, IPG measures the electrical resistance of the lower body resulting from changes in body water and peripheral blood flow. To measure IPG signals, the measurement device 100 may be configured with one or more electrical signal sensors 210 that apply a small, varying current to the heels of the user. The IPG signal reflects the electrical resistance of the lower body. In some embodiments, IPG signals are detected using a tetrapolar arrangement of four electrodes 210—two current applying electrodes and two receiving electrodes. The two current applying electrodes may be used to apply a current across the feet of the user while the two receiving electrodes may be used to measure the return signals between the feet of the user. The current travels through the body while the pulsatile flow of blood through the user's legs presents a varying resistance to the applied current. Because blood is a conductive medium, the resistance is modulated by changes in blood flow, such that the cardiovascular monitoring platform 110 is able to derive biomarker measurements reflecting cardiovascular function, such as pulse rate and peripheral perfusion. The non-pulsatile component of the measured impedance reflects the fluid content of the user's tissues. In some embodiments, the IPG signal is captured at multiple frequencies, so that the cardiovascular monitoring platform 110 determines impedance biomarkers that reflect intra-cellular and extra-cellular fluid levels. In some embodiments, the measurement device 100 captures an IPG signal at two frequencies where the lower frequency is representative of the extracellular fluid levels and the higher frequency is representative of total fluid levels. In other embodiments, the measurement device 100 captures the IPG signal at multiple frequencies and applies bioimpedance spectroscopy techniques to derive the fluid level biomarkers.

The BCG signal measures the cyclical hemodynamic forces transmitted from the heart with each cardiac systolic ejection. Described differently, BCG signals are indicative of the mechanical function of the heart and blood flow through the aorta. BCG signals may be measured through the small forces imparted on the body with each heartbeat. To measure BCG signals, the measurement device 100 may be configured with one or more force sensors 230 positioned to detect the weight exerted on the device 100. The force sensors 230 may measure the weight of the user standing on the device 100 and the dynamic forces, such as the BCG, exerted on the device 100 by the user. In some embodiments, BCG signals are measured using the sensors 230 used for weight measurement. Accordingly, the BCG signal reflects small forces exerted on the body by each cardiac contraction and blood flow through the aorta and captured by the measurement device 100.

To measure ECG signals, the measurement device 100 may be configured with electrical signal sensors to detect an electrical signal between the user's heels when the user stands on the device 100. In some embodiments, ECG signals are measured using the same electrodes/sensors 210 used to measure the IPG signal. A pair of electrical signal sensors 210 measure the potential difference generated across the body of the user that reflects the electrical activity of the heart. In some embodiments, four ECG electrodes may be used to capture the multiple ECG signals. The cardiovascular monitoring platform 110 may analyze each ECG signal to derive biomarker measurements. For example, an ECG signal may be collected across the front of the feet and a separate ECG signal across the back of the feet. The cardiovascular monitoring platform 110 may derive one or more biomarker measurements from the highest quality ECG signal collected by the measuring device.

The cardiovascular monitoring platform 110 analyzes the signals received from the sensors 210 and 230 (e.g., the BCG, ECG, and IPG signals described above) to extract biomarkers based on the relationships between characteristic features of different signals or characteristic features within an individual signal. As described herein, a characteristic feature of a signal may be any morphological characteristic or fiducial on the waveform or mathematical function of the signal. Examples of such characteristic features of the signal include, but are not limited to, peaks of a waveform, timings of the peak of the waveform, amplitude of the waveform, frequency content of the waveform, transforms of the signal, derivatives of the signal, integrals of the signal. etc. The cardiovascular monitoring platform 110 may extract features from a time series waveform, a derivate waveform, the frequency transform, or an ensemble average of the signal over multiple heartbeats. As described herein, a biomarker derived from one or more features of one or more signals represents some aspect of cardiovascular function that is clinically interpretable. Described differently, a feature of a signal is a mathematical function or morphological characteristic with limited clinical interpretability, but a biomarker derived from one or more features is a clinically interpretable representation of cardiovascular function. Accordingly, the cardiovascular monitoring platform 110 may generate and offer clinically significant insights to medical providers by analyzing a biomarker derived from features of one or more signal waveforms. Examples of such insights and analysis are further described below with reference to FIG. 3.

In the embodiment of the measurement device 100 illustrated in FIGS. 2A-B, a user must stand on the surface of the device 100. Once the user steps on the scale, the measurement device 100 begins collecting signal data for the user over a short interval, for example 10-30 seconds. In one embodiment, the device 100 notifies the user when it has completed collection of such measurement through a haptic vibration and displays the results (in part or whole) via an integrated visual display. For example, the device 100 may display to the user measurements collected for body weight, peripheral impedance parameters, and pulse rate information via the visual display of the device 100.

The measurement device is described in further detail in U.S. patent application Ser. No. 15/743,154, filed Nov. 2, 2015, and U.S. Pat. No. 11,197,628, filed Oct. 17, 2018, both of which are incorporated by reference herein in their entirety.

In some embodiments (not shown), the cardiovascular monitoring system receives biomarker measurements from data sources other than the measurement device 100. Data collected by secondary data sources is referred to herein as auxiliary biomarker data. In one embodiment, a user may wear an implantable sensor, which collects auxiliary biomarker data and transmits the auxiliary biomarker data to the measurement device 100 and/or the cardiovascular monitoring system 110. For example, the user may wear an implantable pulmonary artery pressure sensor or an implantable electronic cardiac device with embedded sensors. In another embodiment, a user wears a blood pressure cuff while standing on the measurement device 100. The blood pressure cuff collects auxiliary biomarker data simultaneous with the measurement device 100. In embodiments where the cardiovascular monitoring system 110 receives auxiliary biomarker data from a secondary source, the cardiovascular monitoring system 100 assigns a time stamp to each auxiliary measurement and synchronizes each auxiliary measurement with measurements collected by the measurement device 100. In this manner, the cardiovascular monitoring system 100 organizes biomarker measurements collected by the measurement device 100 and auxiliary biomarker measurements recorded during the same time period.

### Cardiovascular Monitoring Platform

The cardiovascular monitoring platform 110 generates a composite biomarker index, referred to hereafter as a heart function index, based on a combination or composite of non-invasive signals and biomarkers measured by the measurement device 100. As described herein, the heart function index 100 characterizes a user's risk of worsening heart failure or a likelihood that a user will experience a heart failure event in the future. The cardiovascular monitoring platform 110 analyzes a combination of biomarkers, for example hemodynamic biomarkers, to output the heart function index. As described herein, the heart function index is a single numerical value that describes the likelihood that the user will experience a heart failure event, or their heart status will worsen. In some embodiments, the heart function index may also characterize the likelihood that a user will be hospitalized due to heart failure. The cardiovascular monitoring platform 110 may further categorize the heart function index into a risk state that indicates the likelihood of a heart failure event occurring within a period of time, for example 30 days or 90 days from the user's use of the measurement device 100. For example, the cardiovascular monitoring system may categorize the heart function index into categories such as “unknown risk state,” “low risk state,” “elevated risk state,” or “high risk state.”

Additionally, the cardiovascular monitoring platform 110 generates an alert that provides a clinician with physiological insight into whether the user's risk state has improved or worsened. For example, the cardiovascular monitoring platform 110 may transmit the heart function index to the clinician. In some embodiments, the generated alert provides additional information regarding the user's predicted future risk of clinical deterioration and/or identifies users at low risk of deterioration.

FIG. 3 illustrates an example system architecture for a cardiovascular monitoring platform 110, in accordance with one or more embodiments. The cardiovascular monitoring platform 110 includes a biomarker data store 310, a biomarker processing module 320, a heart function model 330, a training data set 340, and an alert generation module 350. Computer components such as web servers, network interfaces, security functions, load balancers, failover servers, management and network operations consoles, and the like are not shown so as to not obscure the details of the system architecture. Alternative embodiments may include more, fewer, or different components from those illustrated in FIG. 2, and the functionality of each component may be divided between the components differently from the description below.

The biomarker data store 310 stores signals measured by the measurement device 100 and biomarker measurements derived from the measured signals by the biomarker processing module 320. Biomarker measurements generated by the biomarker processing module 320 are further described below. Additionally, biomarker measurements are stored in the biomarker data store 310 longitudinally such that the biomarker processing module 320 may model and analyze both absolute and relative changes over time in biomarker measurements for a user. In some embodiments, the biomarker data store 310 may be organized by user, such that data associated with each user is stored separately from other users. As described above, signals are collected directly by the measurement device 100, for example IPG, BCG, and ECG signals.

As described above, the measurement device 100 collects signal measurements non-invasively and intermittently over short measurement windows (e.g., 10-30 seconds). Accordingly, biomarkers whose measurements are derived from signal data collected during a single measurement are referred to as “intermittent biomarkers.” Described differently, intermittent biomarkers can be derived from a single measurement. Examples of intermittent biomarkers include, but are not limited to weight, impedance, pulse rate and ultra-short pulse rate variability. However, these short, intermittent measurements result in less data available to the cardiovascular monitoring platform 110, so the cardiovascular monitoring platform 110 derives measurements for biomarkers that would take longer to measure from the collected intermittent measurements. Such biomarkers, referred to as longitudinal biomarkers, are measured over time periods greater than the short measurement window during which the user uses the measurement device 100. Accordingly, such longitudinal biomarkers are derived from multiple measurements collected by the measurement device 100. The biomarker processing module 320 derives longitudinal measurements for a given time period from a series of intermittent measurements collected during the time period, for example by concatenating, averaging, or otherwise processing the multiple intermittent measurements collected during the time period. For example, the biomarker processing module 320 may calculate pulse rate variability using the standard deviation of pulse rate intervals calculated over multiple measurements (e.g., 7 days). Further, the biomarker processing module 320 may characterize the change in an intermittent biomarker as a longitudinal biomarker, for example the change in absolute impedance or weight over multiple measurements or over a specified number of days when measurements were collected by the device. Other longitudinal biomarkers include, but are not limited to, changes in stroke volume, changes in blood pressure, average blood pressure over multiple days, standard deviation of weight from multiple measurements, or any other suitable statistical change in a biomarker over more than one measurement.

As described above, the biomarker processing module 320 extracts (or derives) measurements of particular biomarkers from one or more features of a signal. A feature of a signal can be represented by a morphological characteristic or mathematical function of the signal. Examples of signal features analyzed by the biomarker processing module 320 to derive biomarker measures include, but are not limited to, the time difference between morphological points on two signals, peak amplitudes of a waveform, timings of the peak of the waveform, amplitudes of other waveform features, amplitudes of the derivative of the signals, and frequency content of the waveform. In some embodiments, the biomarker processing module 320 extracts a biomarker measurement from multiple features compared to a reference standard, for example cardiac output. The biomarker processing module 320 computationally derives biomarker measurements from signals measured by the measurement device 100 such that each biomarker characterizes a different clinically relevant aspect of a user's cardiovascular health compared to the signals collected by the measurement device 100. Biomarkers may also be referred to herein as “derived features” because they are biological measurements derived from one or more features of one or more signal measurements. As described above, extracted biomarker measurements have clinical significance that is easily interpretable and understood by medical professionals or clinicians. In contrast with other machine learning systems that make predictions or estimates lacking interpretability (e.g., “black box” models), the heart function model 330 computes heart function indices based on biomarker measurements derived from the signals collected by the measurement device 100 and can be easily analyzed by a medical professional or clinician to identify which biomarkers contributed to the computed heart function index and the extent of their contribution.

Examples of biomarkers extracted from the IPG signal measured by the measurement device 100 include, but are not limited to, peripheral impedance, resistance, reactance, extracellular fluid level, intracellular fluid level, total body water level, phase angle, pulse rate, and pulse rate variability. The biomarker processing module 320 extracts peripheral impedance parameters from the IPG signal measured by the measurement device 100. The magnitude of the IPG signal (i.e., peripheral impedance) is a measure of fluid levels in the lower extremities. In one embodiment, the biomarker processing module 320 correlates a decrease in the magnitude of the peripheral impedance with an increase in risk of a heart failure event. The phase angle of the IPG signal is a measure of musculoskeletal state of the user that may be used to measure loss of lean muscle mass. In one embodiment, the biomarker processing module 320 correlates a decrease in phase angle magnitude with an increase in risk of a heart failure event. The ratio of the impedance magnitude at one frequency to the impedance magnitude at another frequency is a measure of the extracellular fluid in the lower limbs. In one embodiment, the biomarker processing module 320 correlates an increase in the ratio of impedance magnitudes with an increase in risk of a heart failure event. The pulse rate may increase in the event of worsening heart condition as the heart compensates for decreased stroke volume. Accordingly, the biomarker processing module 320 correlates an increase in pulse rate with an increase in risk of a heart failure event.

In one embodiment, the biomarker processing module 320 correlates increases in body weight with an increase in risk of a heart failure event since weight changes may be due to fluid accumulation. Although weight is an indicator of cardiovascular health and daily weight monitoring is recommended as part of heart failure care, weight alone may offer insufficient sensitivity and specificity for detecting worsening heart failure. Accordingly, the biomarker processing module 320 augments the weight signal collected by the measurement device 100 by deriving biomarker measurements from the collected signals that may help identify the underlying reason for the weight change (e.g., edema, dehydration, cachexia, body composition changes, clothing). Accordingly, the biomarker processing module 320 is able to use smaller, earlier weight changes as part of its event prediction, without increasing false positives associated with non-fluid weight changes.

In circumstances where a user gains or loses weight, the biomarker processing module 320 may perform bioelectrical impedance vector analysis (BIVA) to determine whether the change in weight was due to fluid changes or soft tissue changes. A user at risk of heart failure may experience weight loss but that weight loss may be counterbalanced by weight gain due to fluid accumulation. Accordingly, the biomarker processing module 320 may perform impedance vector analysis to determine fluid status and body composition to detect signs of malnutrition and cachexia. Additionally, the biomarker processing module 320 may measure the impedance vector at multiple frequencies to distinguish between changes in extracellular fluid levels and changes in body composition.

The biomarker processing module 320 may derive additional biomarkers from features of the measured signals including, but not limited to, signal quality, pulse rate variability (PRV), signal amplitude, and cardiac time intervals. As described herein, signal quality is a measure of the deviation of signal waveform statistics from the expected statistics of the waveform. For a user with a worsening heart state, the IPG signal and derivative measurements will deform, for example a loss of distinctiveness at the peaks of the signal and/or motion artifacts due to deteriorating postural stability. In some embodiments, the biomarker processing module 320 determines a signal quality measurement based on the median signal quality recorded over the preceding 15 days. In such embodiments, the biomarker processing module 320 correlates a decrease in signal quality with an increase in risk of a heart failure event. In some embodiments, the biomarker processing module 320 may determine a binary indicator of signal quality where the indicator is set to a non-zero constant (e.g., 1) if the signal is low quality and otherwise set to another constant (e.g., 0).

As described herein, PRV is a measure of the variation in heartbeat intervals and may reflect autonomic function and cardiac health. PRV decreases with poor cardiac performance and may be an indicator of an arrhythmia. The biomarker processing module 320 may determine the PRV based on the time between peaks in the derivative of the IPG, BCG, or ECG signal. In one embodiment, the biomarker processing module 320 correlates decreases in PRV with increases in risk of a heart failure event. In some embodiments, the biomarker processing module 320 may determine heart rate variability (HRV) based on the time between peaks in the derivative of the ECG signal. In some embodiments, the biomarker processing module 320 may determine a binary indicator of the presence of an increasing pulse rate trend over the 3 days leading up to the current day where the indicator is set to a non-zero constant (e.g., 1) if the biomarker processing module 320 determines an upward trend in pulse rate and otherwise set to another constant (e.g., 0).

In some embodiments, the biomarker processing module 320 determines the amplitudes of the signals, such as the BCG signal and the IPG signal measured by the measurement device 100. The heart function model 330 may receive signal amplitude measurements to improve the accuracy of the heart function index output. Because amplitude measurements of both the BCG and IPG signals collected by measurement device 100 are correlated to the volume of blood in the aorta and peripheral arteries, the biomarker processing module 320 correlates the maximum amplitude of the BCG signal and the IPG signal with stroke volume (e.g., the amount of blood ejected from the heart with each beat), blood flow, and blood pressure, or another cardiac output biomarker. In such embodiments, the biomarker processing module 320 determines signal amplitude using any suitable signal processing technique, for example RMS power, area under the curve, or by measuring the amplitude of the respective signal. The biomarker processing module 320 may determine the amplitude using an ensemble average of a signal over multiple heartbeats. An ensemble average may result in a more robust biomarker estimation, especially in the presence of noise or artifact. Stroke volume may decrease as heart failure worsens due to the weaking of heart muscle contractions and blood flow to the legs may decrease due to reduced stroke volume and peripheral vasoconstriction. As a result, the biomarker processing module 320 correlates decreases in signal amplitude with increases in risk of a heart failure event.

In some embodiments, the biomarker processing module 320 extracts one or more biomarker measurements from the magnitude of the peripheral impedance described above. In one embodiment, the biomarker processing module 320 determines a change in the magnitude value relative to the user's normal baseline magnitude value. In another embodiment, the biomarker processing module 320 determines a difference between the magnitude value of the user and the value of a reference population. The cardiovascular monitoring platform 110 may generate the reference population by sampling a dataset of other users/users associated with a measurement device 100 to identify other users demographically similar to the user (e.g., age, weight, race, etc.) with a comparable cardiac risk state. In one embodiment, the reference population may be generated by identifying, in part, users with a heart function index in the same category as the user. In another embodiment, the reference population may be generated by identifying, in part, users with a heart function index within a certain range of the user. In some embodiments, the heart function model 330 may generate a heart function index based on an absolute impedance biomarker independent of any longitudinal biomarkers to generate alerts and risk state notification during the user's initial use of the measurement device 100 or their initial period of use.

The biomarker processing module 320 may derive biomarker measurements from morphological features and time periods that occurred within and between the three signals collected by the measurement device 100. In such embodiments, the biomarker processing module 320 may synchronize signal data from different sources to extract biomarkers from relationships between the different signals.

In one embodiment, the biomarker processing module 320 extracts biomarker measurements from temporal information extracted from the BCG, ECG, and IPG signals, for example systolic time intervals. As described herein, systolic time intervals (STIs) are time periods between morphological features of the IPG, ECG, and BCG signals. In one embodiment, the cardiovascular monitoring platform 110 may extract the time difference between morphological features on two different signals to measure STIs that occur between various events within the cardiac cycle. Examples of STIs include pre-ejection period (PEP) and left ventricular ejection time (LVET). The PEP and LVET characterize the contractility and ejection times of the heart. The biomarker processing module 320 measures the PEP in proportion to the time between the QRS complex of the ECG signal and the J wave of the BCG signal. The biomarker processing module 320 may correlate increases in PEP with increased risk of a heart failure event because increases in PEP may result from decreased contractility. The biomarker processing module 320 may also correlate PEP measurements with changes in blood pressure. The cardiovascular monitoring platform 110 may use ratio of PEP/LVET as a biomarker since it is correlated to ejection fraction.

The biomarker processing module 320 may also determine pulse wave transit times that reflect blood pressure and arterial stiffness. For example, the pulse transit time (PTT) is the time period between the J wave of the BCG and the arrival of the pulse wave at the legs of the user standing on the measurement device 100 (e.g., the peak in the IPG signal). As another example, the platform 110 may use the pulse arrival time (PAT) to estimate changes in blood pressure. The PAT is the time period between the QRS complex and the arrival of the pulse wave at the legs of the user (e.g., the peak in the IPG signal). The biomarker processing module 320 determines the PAT based on the sum of the PEP and the PTT. Because the PAT and PTT both also related to the contractility of the heart and the pulse wave velocity, the biomarker processing module 320 correlates both transit times with changes in blood pressure. In particular, the biomarker processing module 320 inversely correlates PAT and PTT with blood pressure. Because blood pressure may increase or decrease prior to a heart failure event, the biomarker processing module 320 may recognize increases or decreases in PAT as an indication of abnormal hemodynamics and heart function decompensation.

The accuracy of the heart function index may increase as the number of biomarker measurements input to the model increases. Accordingly, the biomarker processing module 320 may apply a threshold condition describing a minimum amount of biomarker measurements or a particular subset of biomarkers to be input to the heart function model 330 for the model 330 to generate a heart function index. In one embodiment, a clinician or operator of the cardiovascular monitoring system 110 manually defines the threshold condition. In another embodiment, the biomarker processing module 320 determines the threshold condition based on historical heart function indices generated by the model 330 and the accuracy of each prediction. In such embodiments, the accuracy of each prediction may be characterized based on feedback provided by a clinician, user, or any other suitable entity. If the biomarker processing module 320 determines that measurements for the intermittent biomarkers and longitudinal biomarkers do not satisfy the threshold condition, the biomarker processing module 320 may generate an alert for a clinician to collect additional or missing biomarker measurements or a request for the heart function model 330 to generate a heart function index based on the available biomarker measurements.

The biomarker processing module 320 may generate the same or similar biomarker measurement using multiple distinct signals that each provide insights to the single biomarker measurement. In one embodiment, the biomarker processing module 320 may calculate pulse rate (PR) and/or pulse rate variability using each of the IPG and BCG signals. In embodiments where the IPG and BCG signals may be corrupted by different and independent sources of noise (e.g., poor electrode contact for IPG, motion artifact for BCG), the biomarker processing module 320 may select the higher quality signal for inclusion in the heart function model 330. The biomarker processing module 320 may also increase the confidence in the output of the heart function model 330 if signals from different signal sources are closely correlated. In another embodiment, the biomarker processing module 320 may combine signals from multiple signal sources to compensate if signal quality is low over a portion of one signal. In some embodiments, the biomarker processing module 320 may consider biomarkers that are correlated, but not identical. For example, the biomarker processing module 320 may process the impedance phase angle and impedance ratio together to obtain more robust measurements and reject outliers. Correlated biomarkers may be subject to different or independent sources of noise, artifact, or corruption, making them useful for identifying biomarker quality or corruption.

Signals collected by the measurement device 100 (e.g., BCG, ECG, and IPG signals) and/or biomarkers stored in the biomarker data store 310 may further be categorized into pulse biomarkers, and non-pulse biomarkers. As described herein, pulse biomarkers are derived from the pulsatile or AC portion of a signal modulated by the cardiac cycle (i.e., depolarization, ejection of blood through aorta, blood flow in peripheral body segments). Pulse biomarkers include, but are not limited to, pulse rate, pulse rate variability (PRV), velocity index (VI), PEP, stroke volume, blood pressure. As described herein, non-pulse biomarkers are derived from the non-pulsatile baseline of a signal or DC portion and are not primarily influenced by the pulse signal. Pulse biomarkers characterize time varying processes and may have frequency content that overlaps with a signal artifact, preventing filtering and extraction of the biomarker for some user. In contrast, non-pulse biomarkers may be extracted more easily from a noise corrupted signal and may provide a stronger foundation for a heart function index computation. Non-pulse biomarkers reflect static or low frequency measurements, such as fluid levels or weight. Other non-pulse biomarkers include, but are not limited to, impedance, and phase angle. The biomarker data store 310 may further classify pulse and non-pulse biomarkers as static or dynamic. As described herein, dynamic biomarkers represent the biomarker change over the course of and within a single measurement (e.g., fluid shift after standing in an impedance signal or a change in heart rate) whereas static biomarkers are the average biomarker value over the measurements or a non-changing parameter (e.g., median pulse rate, average body weight). During a time period where measurements for pulse biomarkers are unavailable, the heart function model 330 may generate a heart function index based on available non-pulse biomarkers.

The biomarker processing module 320 determines biomarker baselines to calculate biomarker changes or longitudinal biomarkers. For example, the biomarker processing module 320 may determine a weight baseline and impedance to calculate the Heart Function Index. The biomarker processing module 320 may determine such baselines based on measurements taken during a period when a user's heart function index indicated that they were not at risk of a heart failure event or were at low risk of a heart failure event. For example, the biomarker processing module 320 may determine a user's median weight and median impedance magnitude during said low-risk periods. For users who have not been categorized as either no-risk or low-risk, the biomarker processing module 320 may determine the baselines based on measurements collected or derived during a preceding window of time, for example 30 days, 3 months, or 6 months. In some embodiments, the biomarker processing module 320 may consider more than one candidate baseline and select a baseline with the highest impedance magnitude and lowest weight. In some embodiments, the biomarker processing module 320 may reset and recompute a baseline measurement in response to instructions from a clinician, for example following an intervention.

As discussed above, biomarker data is stored longitudinally in the biomarker data store 310 to model intra-user changes over time. The biomarker processing module 320 computes biomarker baselines to track intra-user changes in the biomarker over time that may be prognostic. The biomarker baseline may be a moving average or median of the biomarker calculated over a longitudinal time window, or some other statistical measure that reflects the biomarker's normal range of values and variability. In some embodiments, the biomarker processing module 320 determine a baseline measurement for one or more biomarkers based on measurements collected for a reference population, for example median impedance magnitude of a reference population.

Alternatively, the biomarker processing module 320 may determine the baseline measurement for one or more biomarkers based on historical measurements collected for the user. In such embodiments, the biomarker processing module 320 may adjust the window from which historical measurements are collected based on the risk state of the user. For example, the biomarker processing module 320 may increase the window for patients in a more severe risk state (e.g., elevated-risk state or alert state) or exclude measurements from elevated-state and/or alert state periods from the window. When the user first begins using the measurement device 100, the biomarker processing module 320 may determine an initial baseline. Because measurements for certain biomarkers may be scarce or inconsistently recorded during the user's initial use of the measurement device 100, the biomarker processing module 320 may determine the initial baseline with an emphasis on certain biomarkers, for example weight measurements and impedance data. In one embodiment, the biomarker processing module 320 may use or rely more heavily on the absolute value of available biomarkers (e.g., absolute impedance magnitude) to determine an initial assessment of the risk state of the user so that the heart function model 330 computes a heart function index after a user's first use of the measurement device 100 or a period of initial use.

In some implementations, the heart function model 330 computes a heart function index based on additional user data collected by a clinician, for example in an inpatient setting or during a hospital admission. If a user is hospitalized or undergoes a significant intervention (e.g., receives IV diuretics), the heart function model 330 may be reset automatically or manually and compute an updated baseline for the user. Accordingly, the biomarker processing module 320 may determine an initial baseline based on historical measurements of the user or historical measurements collected from users with a similar cardiovascular risk state. The biomarker processing module 320 may determine the window of such historical measurements based on the risk state of the user or from a selection of multiple candidate windows. As the measurement device 100 continues to collect and transmit biomarker measurements to the cardiovascular monitoring platform 110, the heart function model 330 considers both absolute and relative biomarker measures to improve the accuracy of the computed heart failure indices, for example changes in impedance and absolute impedance. In other embodiments, the heart function model 330 computes heart failure indices using medication data (e.g., diuretic escalations)

In one embodiment, the biomarker processing module 320 determines biomarker measurements based on changes in signals collected by the measurement device 100 while the user performs a specific action. For example, the biomarker processing module 320 may determine biomarker measurements based on changes in BCG, ECG, or IPG signals collected while the user moves from a seated position to a standing position, undergoes the Valsalva maneuver, or holds their breath while standing on the scale. For example, the biomarker processing module 320 may determine when the patient is seated or standing or when their position changes based on the center of pressure calculated from the load sensors 230 and weight signals. The biomarker processing module 320 may measure the change in a biomarker as the user changes their position to determine a new biomarker measurement. For example, the biomarker processing module 320 may calculate a change in pulse rate during the change in position and relate the magnitude of the change to the user's heart failure risk.

In some embodiments, the biomarker processing module 320 verifies whether measurements collected by the measurement device 100 were collected during a specified time window (e.g., a scheduled time for the user to use the measurement device). The time window may be specified for a particular user or set to a default time by the clinician. In some embodiments, the biomarker processing module 320 does not generate derivative biomarker measurements or input biomarkers to the heart function model 330 unless the signals collected by the measurement device 100 were collected during a specified time window (e.g., between 10 AM and 2 PM). Additionally, the measurements may be excluded from subsequent updates to any baseline measurements. For example, the biomarker processing module 320 may only derive biomarker measurements used in the heart function model 330 from signals collected from uses of the device in the morning, for example measurements collected before 12:00 PM local time.

A user may erroneously or intentionally take multiple measurements within a single time window. In such circumstances, the biomarker processing module 320 only processes the most recent set of measurements taken by the user within the time window to be input to the heart function model 330. Accordingly, upon receipt of a new measurement during a time window, the biomarker processing module 320 deletes any preceding measurements from the same time window from the biomarker data store 310.

In some embodiments, the biomarker data store 310 may store adherence measurements and leverage adherence or non-adherence as a biomarker. For example, the heart function model 330 may predict an increased likelihood of worsening heart condition when the user records fewer measurements than they should. Accordingly, the cardiovascular monitoring platform 110 may implement a feedback loop between engagement and adherence-oriented actions and the heart function index generated by the heart function model 330.

In some embodiments, all signals and biomarkers may not be available from a given measurement or for a given time period, for example due to noise, motion, or early termination of use of the measurement device 100. In such embodiments, the heart function model 330 may still compute a heart function index based on the available biomarkers in a measurement or time period. Even in embodiments where values of all other biomarkers are unavailable, the heart function model 330 may compute a heart function index based on at least a weight measurement. In some embodiments, the heart function model 330 may generate a heart function index for a given time period based on any number of biomarkers as long as a weight measurement has been collected during the given time period.

In some embodiments, the biomarker processing module 320 determines whether to input biomarker measurements and the measured signals to the heart function model 330 to compute a heart function index based on the availability of other biomarker measurements. In one embodiment, the heart function model 330 may not generate a heart function index for a given day if no measurements were collected on that day. In another embodiment, the heart function model 330 may not output a heart function index if no other measurements were collected during a previous time period, for example the previous 15 days. In an embodiment where the only available biomarker for the current day is a body weight measurement (e.g., an impedance measurement is unavailable), the biomarker processing module 320 may impute the most recently collected or derived measurements for biomarkers known to have a correlation between consecutive measurements (e.g., impedance biomarkers).

As described above, the measurement device 100 records measurements for biomarkers and biomarkers in short, intermittent intervals, for example 10-30 seconds. These short measurement intervals reduce the burden on the user to routinely use the measurement device 100 and allow the user to seamlessly integrate the measurement into a form factor and device 100 that is part of their existing habits and/or routines. However, small amounts of noise or artifacts in any portion of the collected biomarker may corrupt the entire measurement. Alternatively, a user may not take measurements on certain days causing there to be missing data for certain days. Accordingly, the biomarker processing module 320 implements one or more of the following techniques to derive additional biomarkers from the biomarkers collected by the measurement device 100. In some embodiments, the biomarker processing module 320 compensates for measurements missed due to low adherence or poor connectivity by estimating (e.g., predicting) or imputing the biomarker value based on past biomarker values. The biomarker processing module 320 may also delete measurements corrupted by noise or artifacts. The biomarker processing module 320 may further apply signal processing techniques to extract biomarkers from short duration measurements or segments of measurements. In some embodiments, a clinician may manually remove certain biomarker measurements from being considered by the heart function model 330 when generating the heart function index, for example suspect measurements.

A person of ordinary skill in the art would appreciate that the particular techniques for processing biomarkers at the cardiovascular monitoring platform 110 described herein are merely illustrative and that the cardiovascular monitoring platform 110 may implement any suitable or relevant signal processing technique to refine collected biomarkers or generate additional biomarkers in addition to those described therein.

As described above, the heart function index is a composite index that combines multiple biomarkers measured by the measurement device 100 and derived from such measured biomarkers into a numerical value representative of a user's current cardiovascular risk state. The user's current cardiovascular risk state is modeled based on biomarkers derived from the signals collected during the user's most recent use of the measurement device 100. As described herein, the heart function index represents a likelihood that the user will experience a heart failure event in the future given their current risk state. In some embodiments, the cardiovascular monitoring platform 110 applies machine-learning techniques or any suitable mathematical or statistical model to determine the heart failure index for a user. The model used to compute the heart function index is referred to herein as a heart function model 330. In some embodiments, the heart function index has a minimum value of 0 and increases as the user's risk state worsens. In embodiments where the heart function model 330 is a mathematical or statistical model, the heart function model computes the heart function index by inputting signals collected by the measurement device 100 and biomarker measurements derived from the collected signals to a suitable algorithm.

In embodiments where the heart function model 330 is a machine-learning model, the cardiovascular monitoring platform 110 trains the machine-learning model to compute a heart function index for a user based on biomarkers derived from the signals measured by the measurement device 100 (e.g., body weight, impedance, pulse rate, etc.).

In embodiments where the cardiovascular monitoring platform 110 is a machine-learning model, the heart function model 330 may be a mathematical function or another more complex logical structure, trained using a combination of biomarkers stored in the training data set 340 to determine a set of parameter values. As will be described further below, the heart function model 330 may be trained based on user-specific biomarker measurements and signals collected by the measurement device 100. In such embodiments, the heart function model 330 is a function for determining a heart function index for a user and the determined parameter values incorporated into the function. “Parameter values” describe the weight associated with at least one of the features of the encoded feature vector.

A machine-learning heart function model 330 may additionally or alternatively trained on data collected from other users. In some embodiments, the model 330 may be periodically or iteratively re-trained a new data is collected from users of the measurement device 100 to improve accuracy of the model 330. The heart function model 330 may be a classifier trained for optimization over objectives such as accuracy using the training data set 340, which is made up of previously measured biomarkers from a large, diverse cohort of users suffering from heart failure. Entries in the training data set 340 may be labeled with known heart failure events. The training data set 340 is organized into preceding time periods such that each entry is labeled with the time period when it was recorded and a known heart function index during the period of time. During training, the heart function model 330 determines parameter values for each biomarker input to the heart function model 330 by analyzing and recognizing correlations between the biomarkers measured during preceding time periods and the heart function index computed for the preceding time period.

The heart function model 330 may be further trained based on weight measurements collected by the measurement device 100. For example, because the user's weight may increase in advance of a heart failure event, a weight increase may be used to identify and label a heart failure event in the training data set 340. Such labels may be used to train the heart function model 330 using other biomarkers that may change in advance of the weight biomarker. Any of these model training techniques may be used independently or in combination.

As the heart function model 330 computes heart failure indices, which may be verified by operators reviewing the computed indices, the training data set 340 may be continuously updated with entries pertaining to new time periods. In addition, the training data set 340 may be continuously updated as the parameters assigned for certain biomarkers change, for example due to improvements or deteriorations in the cardiovascular health of the user. Accordingly, the heart function model 330 may be iteratively trained based on the updated data in the training data set 240 to continuously improve the accuracy of heart failure indices output by the heart function model 330. For example, a heart failure event may be evaluated and verified by cardiologists or other experts in the field and may be labeled as a heart failure event for future model training based on the evaluation/verification.

In some embodiments, the heart function model 330 comprises one or more sub-models that generate a function index based on a particular combination or category of biomarkers. For example, the heart function model 330 may comprise a congestion sub-model that computes a congestion index based on biomarkers including, but not limited to, bioimpedance vector analysis (described above), phase angle, weight, impedance, correlates to and measures of intravascular volume, and correlates to and measures of filling pressure. The congestion index output describes an assessment euvolemia (e.g., fluid balance) and/or absolute fluid levels based on an established dry weight. The congestion sub-model may enable titration of diuretics. In some embodiments, the congestion submodel computes a congestion index as a composite of measurements for a subset of biomarkers determined to most effectively represent fluid accumulation. In one embodiment, the congestion submodel determines the composite index based on weight measurements (e.g., an increase in a user's weight from their baseline weight), changes in impedance magnitudes (e.g., a decrease in the height-normalized impedance magnitude from the patient's baseline impedance magnitude), and absolute impedance magnitudes (e.g., an amount by which the patient's height normalized impedance magnitude is lower than a median reference heart failure population established using data from prior studies).

The alert generation module 350 generates an alert if the computed congestion index exceeds a previously defined threshold. In some embodiments, the alert generation module 350 implements a global threshold that is the same for every patient in a cohort of patients. In other embodiments, the alert generation module 350 determines user-specific baseline weight and impedance magnitudes using the patient's N-day moving median weight and M-day moving median impedance magnitude. In some embodiments, such moving medians may be calculated using only periods when the patient's congestion index was below the predefined threshold. In one embodiment, N and M are set to 90. The alert generation module 350 may reset an alert in response to the earlier of the computed congestion index decreasing below the global threshold or a period of time after the alert generation (e.g., 30 days). In some embodiments, the congestion submodel may implement an upper threshold above which a congestion alert is generated. As described herein, a congestion alert may predict a heart failure event due to worsening fluid status or fluid accumulation. The congestion submodel may also have a lower threshold below which a user may be too dry or overly diuresed. If the congestion index passes below this threshold, a separate alert may be generated.

As another example, the heart function model 330 may comprise a perfusion sub-model that computes a perfusion index based on biomarkers including, but not limited to, perfusion marks such as pulse rate and pulse rate variability (described above), stroke volume, cardiac output, central mean arterial pressure, left ventricular ejection time, and pre-ejection period. As described herein, central mean arterial pressure (cMAP) is the average pressure in a user's aorta during a single cardiac cycle. Left ventricular ejection time describes the time it takes for blood to be ejected from the left ventricle between the opening and closing of the aortic valve. As a user's risk state worsens, the left ventricle has difficulty producing the contractile force necessary to keep the aortic valve open, which results in decreased left ventricular ejection time and increased isovolumic contraction times. The biomarker processing model 320 may determine the left ventricular ejection time by based on points along the IPG signal representing the opening and closing of the aortic valve. The biomarker processing module 320 may determine the pre-ejection period based on the time interval between the start of electrical depolarization and the start of ventricular ejection, which represents ventricular contraction while the aortic valve is closed. Users at risk of a heart failure event, the pre-ejection period may be prolonged.

In other embodiments, the heart function model 330 is structured as an additive model to compensate for a lack of ground truth hospitalization events. Such additive models may be trained using any suitable techniques, for example backfitting.

In some embodiments, the heart function model 330 computes a heart function index based on biomarkers derived from weight and impedance measurements. For example, one or more biomarkers may be extracted from the weight measurements and one or more biomarkers may be extracted from the impedance measurements. Some biomarkers may be based on the current weight or current impedance measurements of the users while other biomarkers can be based on past weight or impedance measurements of the users. In one embodiment, the biomarker processing module 320 generates user baseline features based on past measurements such that the user baseline features can be used to train the heart function model 330. In one particular embodiment, the heart function model 330 can be a linear or affine function of such user baseline features and biomarkers.

In some embodiments, the heart function model 330, or another suitable model, may verify the accuracy of a heart function index computed by the heart function model 330. For example, a weight measurement collected by the measuring device 100 may be inaccurate if a patient is moving excessively during the measurement or if the patient leans on a weight-bearing object during the measurement. In such embodiments, the biomarker processing module 320 may apply the artifact detection and signal described above. In one embodiment, the biomarker processing module 320 may compute a weight signal from data collected by load cells on the measurement device 100 and identify regions of the signal with high variability as corrupted by the user's motion. The biomarker processing module 320 may additionally derive an EMG signal collected by the ECG electrodes (electrical activity of leg muscles during standing) with a larger amplitude than the ECG signal and identify regions of the EMG signal with a high amplitude as corrupted by the user's motion. The biomarker processing module 320 combines the motion-corrupted regions of both the weight signal and the EMG signal to determine the final motion-corrupted regions during the measurement.

In another embodiment, a biomarker measurement other than weight may be corrupted by noise or artifact. For example, the impedance measurement may require contact between the sensor electrodes and the bare feet of the user, so the impedance measurement may be inaccurate if the user does not make sufficient contact with the electrodes (e.g., wearing socks, incorrect foot positioning). In one embodiment, the biomarker processing module 320 identifies an inaccurate impedance measurement by comparing the measurement against a normal value for a representative population. For example, the biomarker processing module 320 may identify the impedance measurement as inaccurate if it is outside a specific range (e.g., 200-1000 ohms). In another embodiment, the biomarker processing module 320 identifies an inaccurate impedance measurement by comparing it to another impedance measurement collected during the same time period. If the two measurements are not within a certain range of each other (e.g., 10 ohms), the biomarker processing module 320 may identify the measurement as invalid. In another embodiment, the biomarker processing module 320 may identify an impedance measurement as invalid by examining its complex components (e.g., phase angle, reactance, resistance) and comparing them against population-derived thresholds. For example, the biomarker processing module 320 may identify a measurement as invalid if the phase angle is greater than zero. The biomarker processing module 320 may identify other measured signals or biomarker measurements as invalid using any other suitable techniques.

In embodiments where a biomarker measurement or signal measurement is determined to be invalid, the biomarker processing module 320 may remove the measurement and recalculate the heart function index using the remaining input biomarker measurements and signals. In other embodiments, the biomarker processing module 320 may impute a valid biomarker from a prior measurement to be used in the computing the current heart function index. In other embodiments, the biomarker processing module 320 may replace the inaccurate measurement with a mean, median, or weighted average of the biomarker measurement from previous measurements. In most embodiments, the variety of signal sources with difference potential sources of noise (e.g., poor electrode contact vs improper weight distribution) will yield at least one usable biomarker for the heart function index to make a prediction.

The alert generation module 350 generates an alert based on the heart function index computed by the heart function model 330. Based on the absolute value of the heart function index and changes between a current heart function index and a preceding index, the alert generation module 350 categorizes a user into a risk state. As described herein, a risk state represents a range of heart function indexes labeled based on the user's risk of worsening heart failure or a heart failure event, for example low-risk, elevated-risk, or alert. Additionally, the heart function model 330 may compare a current heart function index value to the most recent previous heart function index value to determine whether a user's risk state has worsened or improved. In one embodiment, the alert generation module 350 categorizes a user into one of three states: low-risk, elevated risk, and an alert state. In some embodiments, the alert generation module 350 may be further categorized into an unknown state if the heart function model 330 has not generated a heart function index for a given day and the user does not meet the criteria to be placed in the alert state. In some embodiments, the heart function model 330 computes a daily heart function index for a user (provided that new signals were measured by the measuring device during that day). Accordingly, the heart function index updates a patient's risk state daily (e.g., high risk state, low risk state, alert state).

In such embodiments, the low-risk state and the elevated-risk state are separated by a risk threshold and the elevated-risk state, and the alert risk state are separated by an alert threshold. The risk threshold is a value above which the user enters the elevated-risk state. Users categorized into the elevated-risk state may be at an increased risk of a heart failure event during the next 90 days. An alert is generated and transmitted to the appropriate users and clinicians when the alert generation module 350 categorizes a user into the alert state. In one embodiment, the alert generation module 350 determines a recommended alert threshold based on measurements collected from the reference population or measurements collected for the particular user. In alternate embodiments, the alert generation module 350 receives an alert threshold provided by a clinician. The alert reset threshold is an index value that, if satisfied, triggers the alert generation module 350 to transition the user from the alert state to the low-risk state. The alert generation module 350 may set the reset threshold below the alert threshold to reduce the likelihood of exiting the alert state prematurely. In one embodiment, the reset threshold is an index value below the median heart function index over a period of days prior to when the alert was generated. In other embodiments, the alert generation module 350 may define the alert reset threshold as a function of the heart function index, for example increasing the reset threshold for a lower heart function index compared to a higher heart function index.

The alert generation module 350 may categorize a user into the low-risk state if their heart function index is below a given risk threshold. The alert generation module 350 may transition the user from the unknown or low-risk state to the elevated-risk state if the heart function index exceeds the risk threshold. The alert generation module 350 may transition the user from the unknown, low-risk, or elevated-risk state to the alert state if the heart function index increases beyond the alert threshold within a preceding time period, for example 30-days. The alert generation module 350 may transition a user from the alert state to the elevated-risk state if the heart function index drops below the reset threshold and/or the user has been in the alert state for a threshold amount of time, for example 30 days. The alert generation module 350 may transition a user from the alert state to the low-risk state if the heart function index drops below the reset threshold and below the risk threshold. The alert generation module 350 may transition a user from the elevated-risk state to the low-risk state if the heart function index drops below the risk threshold or if no measurement is taken within a specified time period. The alert generation module 350 maintains a user in the alert state if no measurement is taken during the specified time period and user has been in the alert state for less than a threshold amount of time, for example 30 days.

When the heart function model 330 computes a heart function index for a user already categorized in the alert state and the computed heart function index is greater than the previously computed index (e.g., the user's risk state is worsening), the alert generation module 350 may generate an additional alert to let the user and/or clinician known that their condition has worsened. In some embodiments, the alert generation module 350 generates an additional alert if the computed heart function index is greater than the previous heart function index by an amount equivalent to the alert threshold.

In some embodiments, the alert generation module 350 considers the rate at which the user's heart function index is increasing. If the user enters the elevated risk state or alert category at a rate above a threshold (e.g., faster than anticipated), the alert generation module 350 transmits a notification to a client device of the clinician, the user, or both. For example, if the user's heart function index rises to a value in the elevated risk or alert category at a rate above a threshold rate, the alert generation module 350 transmits a notification to a client device of the clinician, the user, or both.

If the user is already in the elevated risk or alert category and the index continues to increase at a rate above a threshold, the alert generation module may transmit a notification to a client device or the clinician, or user, or both. A clinician may manually define or reset thresholds for the risk categories and alerts, for example to adjust the sensitivity (e.g., the ranges of heart failure indices describing each category) and alert rate. Additionally, a clinician may raise or lower the sensitivity of the heart function model 330 to strike a balance between model sensitivity and specificity or alert burden. As described above, the heart function model 330 generates a heart function index based on, at least, a weight measurement derived from signals collected by the measurement device 100. In one embodiment, the alert generation module 350 generates a notification when a user experiences more than a 3-pound change over 24 hours or a 5-pound change over a 1-week period.

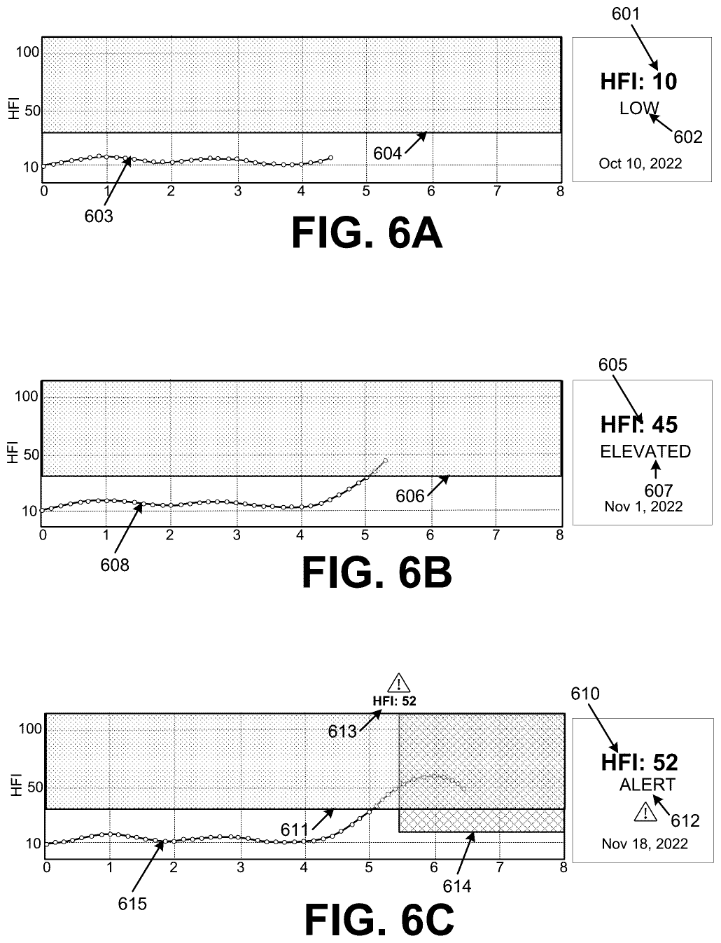

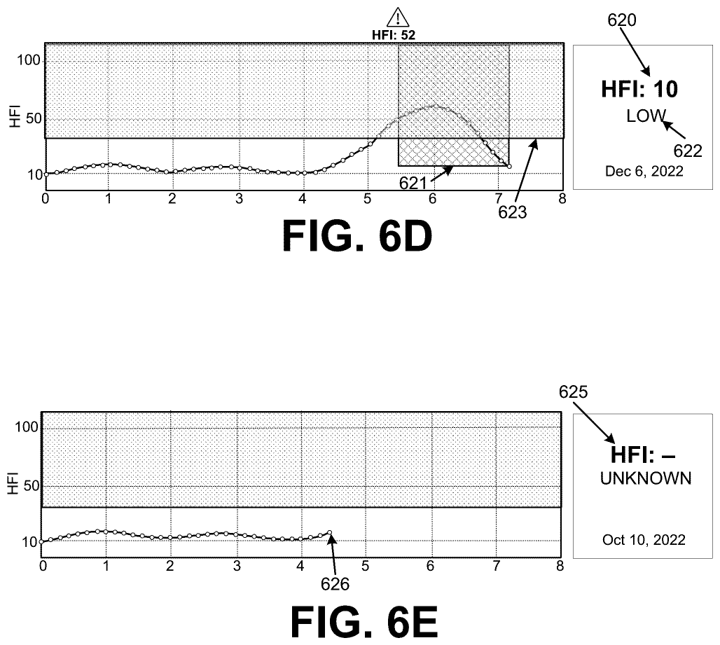

In some embodiments, the alert generation module 350 generates a notification that is easily interpretable by presenting the individual biomarkers underlying the generated heart function index and the value of each biomarker. In some embodiments, the alert identifies the value of each biomarker considered in the generation of the heart function index. For example, the alert generation module 350 may make available to the clinician the value of an individual biomarker that is a component of the heart function index, its trend over a preceding period of time (e.g., a change in the value of the biomarker over a period of time), and the relative contribution of the biomarker value to the heart failure index. The alert may additionally display the contribution of each biomarker to the overall heart function index value as a percentage or fraction. Illustrative examples of the notification generated by the alert generation module 340 are described below with reference to FIGS. 6A-D.

The cardiovascular monitoring platform 110 and clinicians may use the heart function index to predict and avoid hospitalization events, evaluate a user's risk of worsening heart failure or hospitalization, increase the user's adherence, provide updated treatment recommendations, and target diseases other than heart failure. In some embodiments, the cardiovascular monitoring platform 110 leverages the heart function index to automate therapy management, for example medication dosing and titration. For example, the alert generation module 350 may leverage the congestion index predicted by a sub-model of the heart function model 330 to guide diuretic dosing. In such circumstances, the alert generation module 350 may provide a sample workflow or protocol of a biomarker-guided diuretic management.

The heart function model 330 may additionally identify a response of a user to diuretic (e.g., a minimum effective dose and a ceiling dose). In one embodiment, the heart function model 330 creates a user-specific diuretic dose-response curve based on a minimum effective dose and a ceiling dose and diuretic doses received by the user. Accordingly, alert generation module 350 may recommend a diuretic dose to the user or for self-management of diuretics based on the predicted heart function index or automate titration of the recommended dose to the user. In another embodiment, the alert generation module 350 directs the user to IV-diuretic therapy to help prevent or reduce hospitalizations. The alert generation module 350 may use the biomarker contributions to the heart function index and the heart function index overall response to IV-diuretic administration for a particular user to improve the heart function index and/or optimize medication dosing recommendations. In other embodiments, the alert generation module 350 may leverage the predicted heart function index to generate a patient-specific titration protocol, automate pill dispensing protocols, or any other suitable protocol for managing the user's cardiac health.

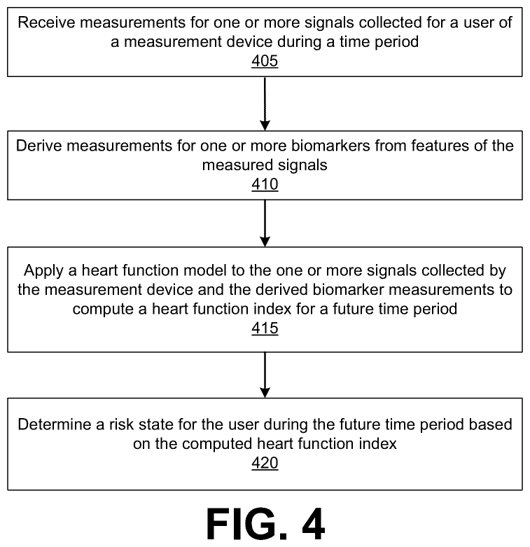

FIG. 4 is a flowchart illustrating a process for determining a heart function index, according to one or more embodiments. The cardiovascular monitoring system 110 receives 405 measurements for one or more signals collected for a user of the measurement device during a time period. As described above, the user stands on the measurement device such that electrodes and load sensors integrated into the measurement device collect electrical signals through the feet of the user and weight measurements for the user. The cardiovascular monitoring system 110 derives 410 measurements for one or more biomarkers from features of the measured signals. As described above, signal features can be represented as mathematical functions of the signal, while biomarker measurements represent biological measurements of clinical significance to medical providers.

The cardiovascular monitoring system 110 applies 415 a heart function model to the one or more signals collected by the measurement device and/or the derived biomarker measurements to compute a heart function index for a future time period. As described above, the heart function index characterizes a likelihood that the user will experience a heart failure event during the future time period. The cardiovascular monitoring system 110 determines 420 a risk state for the user during the future time period based on the computed heart function index. The model may be iteratively re-trained as signals are collected and biomarker measurements are generated for the user and added to the updated training data set 340.

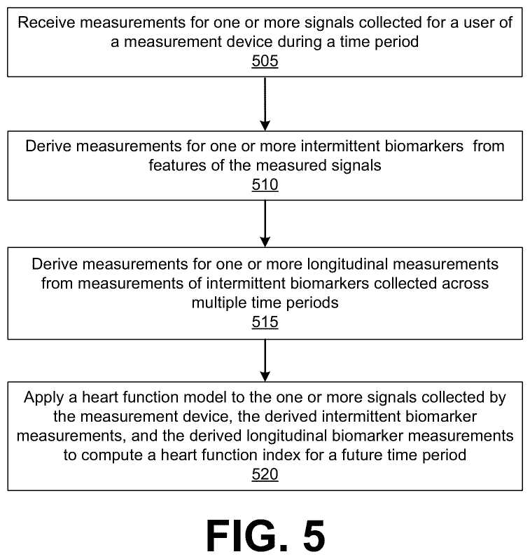

FIG. 5 is a flowchart illustrating a process for deriving biomarker measurements for determining a heart function index, according to one or more embodiments. A cardiovascular monitoring system 110 receives 505 measurements for one or more signals collected for a user of the measurement device during a time period. The cardiovascular monitoring system 110 derives 510 measurements for one or more intermittent biomarkers from features of the measured signals. As described above, intermittent biomarkers are biomarkers whose measurements are derived from signal data collected during a single measurement. The cardiovascular monitoring system 110 derives 515 measurements for one or more longitudinal measurements from measurements of intermittent biomarkers collected across multiple time periods. As described above, longitudinal biomarkers, are measured or calculated over time periods greater than the short measurement window during which the user uses the measurement device 100. Accordingly, the cardiovascular monitoring platform 110 derives longitudinal measurements for a given time period from a series of intermittent measurements collected during the time period, for example by concatenating, averaging, or otherwise processing the multiple intermittent measurements collected during the time period. The cardiovascular monitoring system 110 applies 520 a heart function model to the one or more signals collected by the measurement device, the derived intermittent biomarker measurements, and the derived longitudinal measurements to compute a heart function index for a future time period.

FIGS. 6A-D illustrate example interfaces of notifications displayed to a clinician or a user based on measurements collected by the measurement device, in accordance with one or more embodiments. As described above, the cardiovascular monitoring platform 110 generates notifications displaying a heart function index calculated for a user of the measurement device 100. In some embodiments, the notification additionally displays the risk state for the user based on the displayed heart function index. In some embodiments, the notification further displays the trend in heart function indices predicted for the user over a period of time. FIG. 6A illustrates a notification generated in response to a heart function index generated for a user in a low-risk state, in accordance with one or more embodiments. In the illustrated embodiment, the notification displays a heart function index 601 of “10” determined for the user of, which is lower than the predefined risk threshold 604 of “35.” Accordingly, the user is categorized in the “low-risk” state 602. Additionally, the notification displays a trend 603 of the user's heart function index, which illustrates that the user has been in the low-risk state since they began using the measurement device 100.

FIG. 6B illustrates a notification generated in response to a heart function index generated for a user in an elevated-risk state, in accordance with one or more embodiments. In the illustrated embodiment, the notification displays a heart functions index 605 of “45” determined for the user, which is greater than the risk threshold 606. Accordingly, the user is categorized in the “elevated-risk” state 607. Additionally, the notification displays a trend 608 of the user's heart function index, which illustrates that the user was in the low-risk state until the date when the current heart function index was determined—Nov. 1, 2022.

FIG. 6C illustrates a notification generated in response to a heart function index generated for a user in an alert state, in accordance with one or more embodiments. In the illustrated embodiment, the notification displays a heart function index 610 of “52” determined for the user, which is greater than the risk threshold 611. The heart function index 610 is also greater than a defined alert threshold (not shown), which is sufficient to categorize the user into the “alert” state 612. When the user first entered the alert state 612, the cardiovascular monitoring system 110 generated and transmitted an alert. The interface identifies the date when the alert was generated and transmitted using the flag 613. As described above, when a user enters the alert state, the cardiovascular monitoring system 110 adjusts the reset threshold 614 to a value lower than the risk threshold between the low-risk state and the high-risk state. In the illustrated interface, the reset threshold is defined at an index value of 12. Additionally, the notification displays a trend 615 of the user's heart function index, which illustrates that the user's risk state worsened before improving after entering the alert state 612 but has not yet improved enough to satisfy the reset threshold 614.

FIG. 6D illustrates a notification generated in response to heart function index satisfying the reset threshold value, in accordance with one or more embodiments. In the illustrated embodiment, the notification displays a heart function index 620 of “10” determined for the user, which satisfies the reset threshold 621 of “12.” Accordingly, the cardiovascular monitoring system 110 categorizes the user into the “low-risk” state 623 and re-implements the initial risk threshold 623 of “35.”

FIG. 6E illustrates a notification generated for a user categorized in the unknown state, in accordance with one or more embodiments. In the illustrated embodiment, the measurement device 100 collected no measurements for the user and no heart function index can be calculated for the current day. Thus, the notification lists the heart function index 625 as “unknown” on the current day 626. The cardiovascular monitoring system categorizes the risk state of the user as “unknown” for the current day 626.

### Other Considerations

The foregoing description of the embodiments has been presented for the purpose of illustration; it is not intended to be exhaustive or to limit the scope of the disclosure. Many modifications and variations are possible in light of the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one or more embodiments, a software module is implemented with a computer program product comprising one or more computer-readable media containing computer program code or instructions, which can be executed by a computer processor for performing any or all of the steps, operations, or processes described. In one or more embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor comprises one or more processors or processing units that, individually or together, perform the steps of instructions stored on a computer-readable medium.

Embodiments may also relate to an apparatus for performing the operations herein. This apparatus may be specially constructed for the required purposes, or it may comprise a computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The weights may be stored on one or more computer-readable media, and are used by a system when applying the machine-learning model to new data.

The language used in the specification has been principally selected for readability and instructional purposes, and it may not have been selected to delineate or circumscribe the inventive subject matter. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or.” For example, a condition “A or B” is satisfied by any one of the following: A is true (or present) and B is false (or not present), A is false (or not present) and B is true (or present), and both A and B are true (or present). Similarly, a condition “A, B, or C” is satisfied by any combination of A, B, and C having at least one element in the combination that is true (or present). As a not-limiting example, the condition “A, B, or C” is satisfied by A and B are true (or present) and C is false (or not present). Similarly, as another not-limiting example, the condition “A, B, or C” is satisfied by A is true (or present) and B and C are false (or not present).

What is claimed:

1. A method comprising:
receiving, from a measurement device, one or more signals collected for a user of the measurement device during a time period, wherein the one or more signals comprising a weight measurement for the user and electrical signals collected through the feet of the user;
extracting measurements for a plurality of biomarkers based on the one or more signals collected for the user, wherein each biomarker characterizes an aspect of cardiovascular health of the user;
applying a heart function model to one or more of (1) the one or more signals collected by the measurement device and (2) the plurality of biomarkers, wherein the heart function model outputs a heart function index that characterizes a likelihood whether the user will experience a heart failure event during a future time period; and
generating, for transmission to a computing device of a clinician, an alert based on the heart function index, wherein the alert comprises a risk state of the user, the risk state determined based on a comparison of the heart function index to a threshold.

2. The method of claim 1, wherein the one or more signals are collected by a plurality of electrical sensors and one or more load sensors integrated into the measurement device as the user stands on the measurement device, the one or more signals comprising one or more of the following:
a weight measurement for the user;
an impedance plethysmograph signal;
a ballistocardiograph signal; and
an electrocardiograph signal.

3. The method of claim 1, wherein extracting measurements for the plurality of biomarkers based on the one or more signals collected for the user comprises:
identifying one or more features of each signal of the one or more signals collected by the measurement device, wherein a feature represents a characteristic or function of the signal; and
extracting a biomarker measurement from one or more features of the one or more signals collected by the measurement device, wherein the biomarker measurement is an aspect of cardiovascular health interpretable by a medical professional.

4. The method of claim 1, wherein extracting measurements for the plurality of biomarkers based on the one or more signals collected for the user comprises:
determining measurements for one or more intermittent biomarkers from the one or more signals collected by the measurement device, wherein each intermittent biomarker can be derived from signals collected during a single use of the measurement device; and
determining measurements one or more longitudinal biomarkers based on measurements for an intermittent biomarker collected over different time periods, wherein each longitudinal biomarker represents a measurement collected over a time period greater than the single use of the measurement device.

5. The method of claim 1, further comprising:
for each of the plurality of biomarkers, determining a baseline measurement based on one or more of the following:
measurements collected for the user during a period when a heart function index computed for the user indicated a low likelihood that the user would experience a heart failure event; or
historical measurements collected for the user during a preceding time period.

6. The method of claim 1, wherein the heart function model is a machine-learning model, the heart function model trained based on a training data set of biomarker measurements collected for a population of users, each entry of the training data set labeled with a heart failure event.

7. The method of claim 6, wherein the training data set is periodically updated with biomarker measurements and heart function indexes computed for subsequent time periods and the heart function model is periodically retrained based on the updated training data set.

8. The method of claim 1, wherein the heart function model comprises a congestion sub-model that computes a congestion index representing a fluid status for the user based on measurements collected for a subset of biomarkers characterizing fluid accumulation.

9. The method of claim 1, further comprising:
determining an accuracy of a biomarker measurement based on the signals collected by the measurement device corresponding to the biomarker measurement, wherein signals corresponding to inaccurate biomarker measurements contain regions affected by noise, movement during use of the measurement device, or early termination of use of the measurement device; and
responsive to determining the biomarker measurement is inaccurate, removing the biomarker measurement from the plurality of biomarker measurements input to the heart function model.

10. The method of claim 1, wherein the alert further comprises a graphic representation of a trend of heart function indices predicted relative to a risk threshold for entering an elevated-risk state and an alert threshold for entering an alert state.

11. The method of claim 1, further comprising:
determining the risk state based on comparison of the heart function index to a preceding heart function index.

12. The method of claim 1, further comprising:
comparing the determined heart function index to ta first threshold;
determining a rate of change of the heart function index; and
responsive to determining the heart function index exceeds the first threshold, generating, for transmission, an alert to the user or the clinician, the alert comprising the determined heart function index, the rate of change of the heart function index, and the risk state determined for the user.

13. A non-transitory computer-readable storage medium comprising stored instructions, which when executed by at least one processor, cause the processor to:
receive, from a measurement device, one or more signals collected for a user of the measurement device during a time period, wherein the one or more signals comprising a weight measurement for the user and electrical signals collected through the feet of the user;
extract measurements for a plurality of biomarkers based on the one or more signals collected for the user, wherein each biomarker characterizes an aspect of cardiovascular health of the user;
apply a heart function model to one or more of (1) the one or more signals collected by the measurement device and (2) the plurality of biomarkers, wherein the heart function model outputs a heart function index that characterizes a likelihood whether the user will experience a heart failure event during a future time period; and
generate, for transmission to a computing device of a clinician, an alert based on the heart function index, wherein the alert comprises a risk state of the user, the risk state determined based on a comparison of the heart function index to a threshold.

14. The non-transitory computer-readable storage medium of claim 13, wherein instructions for extracting measurements for the plurality of biomarkers based on the one or more signals collected for the user further cause the processor to:
identify one or more features of each signal of the one or more signals collected by the measurement device, wherein a feature represents a characteristic or function of the signal; and
extract a biomarker measurement from one or more features of the one or more signals collected by the measurement device, wherein the biomarker measurement is an aspect of cardiovascular health interpretable by a medical professional.

15. The non-transitory computer-readable storage medium of claim 13, wherein instructions for extracting measurements for the plurality of biomarkers based on the one or more signals collected for the user further cause the processor to:
determine measurements for one or more intermittent biomarkers from the one or more signals collected by the measurement device, wherein each intermittent biomarker can be derived from signals collected during a single use of the measurement device; and
determine measurements one or more longitudinal biomarkers based on measurements for an intermittent biomarker collected over different time periods, wherein each longitudinal biomarker represents a measurement collected over a time period greater than the single use of the measurement device.

16. The non-transitory computer-readable storage medium of claim 13, further comprising instructions that cause the processor to:
for each of the plurality of biomarkers, determine a baseline measurement based on one or more of the following:
measurements collected for the user during a period when a heart function index computed for the user indicated a low likelihood that the user would experience a heart failure event; or
historical measurements collected for the user during a preceding time period.

17. The non-transitory computer-readable storage medium of claim 13, wherein the heart function model comprises a congestion sub-model that computes a congestion index representing a fluid status for the user based on measurements collected for a subset of biomarkers characterizing fluid accumulation.

18. The non-transitory computer-readable storage medium of claim 13, further comprising instructions that cause the processor to:
determine an accuracy of a biomarker measurement based on the signals collected by the measurement device corresponding to the biomarker measurement, wherein signals corresponding to inaccurate biomarker measurements contain regions affected by noise, movement during use of the measurement device, or early termination of use of the measurement device; and
responsive to determining the biomarker measurement is inaccurate, remove the biomarker measurement from the plurality of biomarker measurements input to the heart function model.

19. The non-transitory computer-readable storage medium of claim 13, further comprising instructions that cause the processor to:
comparing the determined heart function index to ta first threshold;
determining a rate of change of the heart function index; and
responsive to determining the heart function index exceeds the first threshold, generating, for transmission, an alert to the user or the clinician, the alert comprising the determined heart function index, the rate of change of the heart function index, and the risk state determined for the user.

20. A system comprising:
a measurement device comprising one or more sensors configured to collect signals for a user of the measurement device during a time period, the one or more sensors comprising:
a plurality of electrical sensors configured to collect one or more signals through the feet of the user; and
one or more load sensors configured to collect a weight measurement of the user; and
a non-transitory computer-readable storage medium comprising stored instructions, which when executed by at least one processor, cause the processor to:
receive, from a measurement device, one or more signals collected for a user of the measurement device during a time period, wherein the one or more signals comprising a weight measurement for the user and electrical signals collected through the feet of the user;
extract measurements for a plurality of biomarkers based on the one or more signals collected for the user, wherein each biomarker characterizes an aspect of cardiovascular health of the user;
apply a heart function model to one or more of (1) the one or more signals collected by the measurement device and (2) the plurality of biomarkers, wherein the heart function model outputs a heart function index that characterizes a likelihood whether the user will experience a heart failure event during a future time period; and
generate, for transmission to a computing device of a clinician, an alert based on the heart function index, wherein the alert comprises a risk state of the user, the risk state determined based on a comparison of the heart function index to threshold.
